(19) United States
(12) Patent Application Publication
(10) Pub. No.: US 2017/0319251 A1
(43) Pub. Date: Nov. 9, 2017

(54) SECURE GUIDE DEVICE

(71) Applicant: CLARIANCE, Beaurains (FR)

(72) Inventors: Guy VIART, Saint Leger (FR); Jean Yves LEROY, Campagne-les-hesdin (FR); Brice KRIER, Dainville (FR); Sebastien SHULLER, Strasbourg (FR)

(21) Appl. No.: 15/525,975

(22) PCT Filed: Nov. 9, 2015
(86) PCT No.: PCT/FR2015/053030

§ 371 Date: May 11, 2017

(30) Foreign Application Priority Data
Nov. 14, 2014 (FR) 1460985

(51) Int. Cl. A61B 17/84; A61B 17/88; A61B 17/86; A61B 17/00; A61B 17/70

(52) U.S. Cl. CPC A61B 17/848; A61B 17/8897; A61B 17/86; A61B 17/7082; A61B 2017/00862

(57) ABSTRACT

The secured guiding device for positioning and guiding a bone anchoring screw during a surgical procedure according to the present invention, includes a guide pin having a deformable bifid free end which is elastically deformable and consists of two blades having, on the one hand, a first so-called “closed” configuration obtained, for example, by a manual stress so as to position the blades in the prolongation of the longitudinal axis of the guide pin so that the latter can be introduced into a guide tube for its positioning at the surgical site and, on the other hand, a second so-called “open” configuration so as to position the blades in two opposite directions and substantially perpendicular to the longitudinal axis of the guide pin when the blades are outside the guide tube in order to provide a bone support during the positioning of the anchoring screw.

The present invention relates to a secured guiding device enabling the placing and guiding of a bone anchoring screw during a surgical operation such as, for example, during minimally invasive surgeries.

It is noteworthy that the use of guide-pin type guidance devices is in the process of development in the field of fusion treatment of spinal segment of a spine.

It should be noted that the use of a guiding devices presents risks of driving forward the guide pins in the cancellous bone during the installation of the pedicular screws.

Indeed, the profile of the guide pins at their free end can, during this forward drive, cause injuries to sensitive organs.

To avoid this, the surgeons carry out permanent checks using radiographic images to control the positioning of the guide pin.

The large number of such photographs is detrimental for patients, surgeons and healthcare workers present in the operating room because they are subjected to high doses of X-rays.

The purpose of the secured guiding device according to the present invention is to respond to the above-mentioned disadvantages by preventing the forward movement of the guide pin and thus limiting the number of necessary radiographic images and the resulting exposure to X-rays.

The secured guiding device according to the present invention comprises a guide pin having an elastically deformable bifid free end consisting of two blades having, on the one hand, a first so-called “closed” configuration obtained, for example, by a manual constraint so as to position said blades in the prolongation of the longitudinal axis of said guide pin, allowing the latter to be inserted into a guide tube for its positioning on the surgical site and, on the other hand, a second so-called “open” configuration so as to position said blades in two opposite directions and substantially perpendicular to that of the longitudinal axis of said guide pine when the blades are outside the guide tube in order to ensure a bone support when the anchoring screw is being inserted.

The secured guiding device according to the present invention comprises a guide pin, whose blades have, in the so-called “open” position, a radius of curvature with respect to the longitudinal axis of between 2 and 3 millimeters.

The secured guiding device according to the present invention comprises a guide pin, whose free ends of each blade are in the so-called “open” position, spaced at a distance of between 8 and 12 millimeters.

The secured guiding device according to the present invention comprises a guide pin, whose free ends of each blade respectively have on their outer radius a flat spot which provides an osseous support.

The secured guiding device according to the present invention comprises a guide pin which is made of a super-elastic alloy of the Nitinol type.

The secured guiding device according to the present invention comprises a guide pin whose outer diameter is less than 1.60 millimeters.

The secured guiding device according to the present invention comprises a guide pin whose bifid end has an outer diameter in the so-called “closed” position which is less than that of the guide pin.

The description which follows with reference to the accompanying drawings, given by way of non-limiting examples, will enable a better understanding of the invention, the characteristics which it presents and the advantages which it is capable of providing:

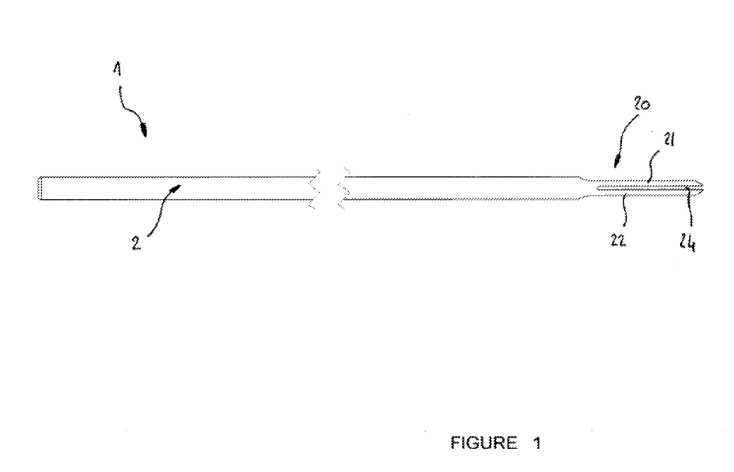

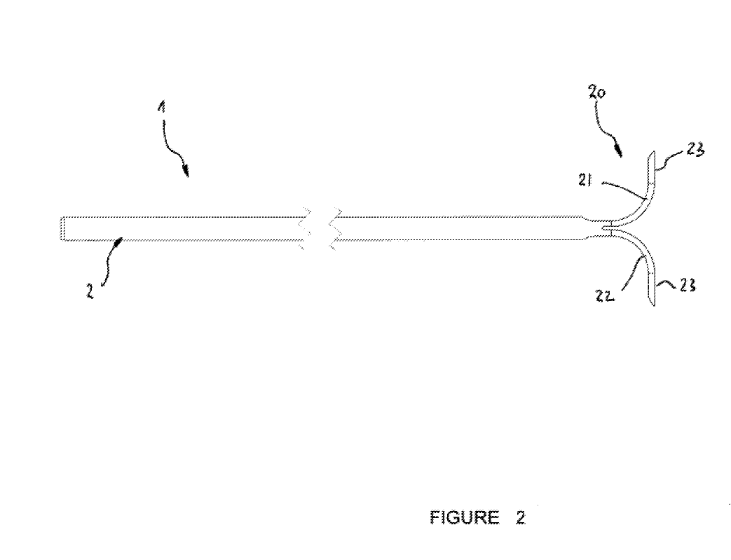

FIGS. 1 and 2 are views illustrating the guide pin of the secured guiding device according to the present invention.

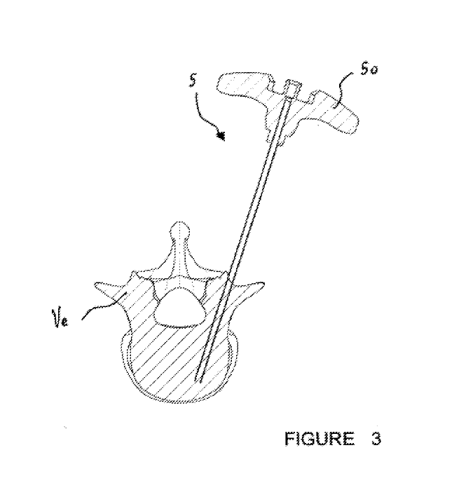

FIGS. 3 to 7 are views showing the positioning in a vertebra of a spinal segment of the guide pin of the secured guiding device according to the present invention.

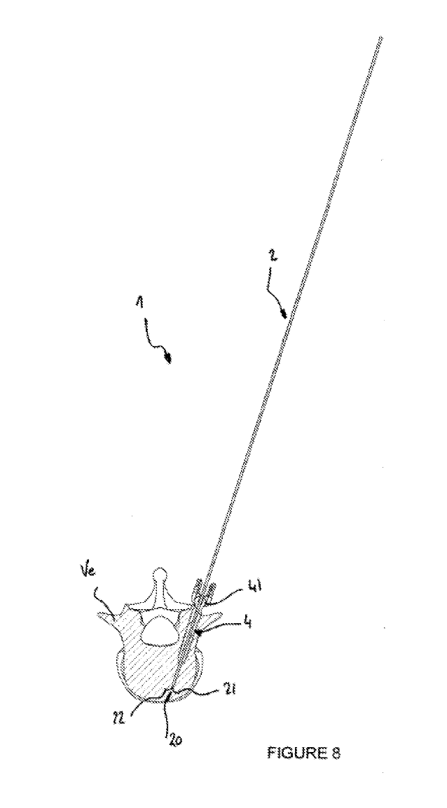

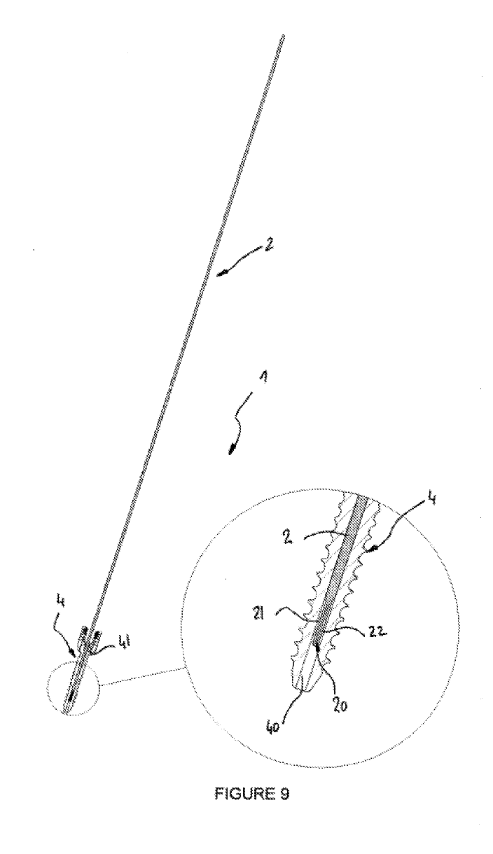

FIGS. 8 to 9 are views showing the guiding of the pedicle screw around the guide pin of the secured guiding device according to the present invention.

FIGS. 1 and 2 show a secured guiding device 1 according to the present invention which consists of a guide pin 2 made, for example, in an elastic alloy of the Nitinol type.

The guide pin 2 has an outer diameter which must be less than 1.60 millimeters in order to be able to slide inside a guide tube 3.

The guide pin 2 comprises an elastically deformable bifid free end 20 constituted by two blades 21, 22 having a first so-called “closed” configuration so as to position said blades 21, 22 in the prolongation of the longitudinal axis of said pin 2 and allowing the latter to be inserted into the guide tube 3 for its positioning in the surgical site.

The two blades 21, 22 of the bifid free end 20 have, due to the elastic characteristics of the guide pin 2, a second so-called “open” configuration so as to position said blades 21, 22 in an opposite direction and substantially perpendicular to that of the longitudinal axis of said pin when the blades are located, for example, outside the guide tube 3, in order to ensure a bone support when an anchoring screw 4 is being placed.

The blades 21, 22 have, in the so-called “open” position, a radius of curvature with respect to the longitudinal axis of the guide pin 2 which is between 2 and 3 millimeters.

It should be noted that the free ends of each blade 21, 22 are in the so-called “open” position, spaced at a distance of between 8 and 12 millimeters.

The free ends of each blade 21, 22 respectively have on their internal face, that is to say those facing each other in the so-called “closed” position, a flat spot 23 ensuring a bone support in the so-called “open” position.

The conjugation of the radius of curvature of the blades 21 and 22 and of the distance between the free ends of said blades in the so-called “open” position allows positioning the flat spots 23 in a plane substantially perpendicular to the longitudinal axis of the guide pin 2.

In the so-called “closed” position, the bifid end 20 of the guide pin 2 has, for example, an external diameter which is smaller than the external diameter of the main body of said guide pin. This decrease in the external diameter of the bifid end 20 is obtained by machining the outer face of the blades 21, 22 in order to reduce the thickness of the latter.

According to another non-limiting example, the bifid end 20 of the guide pin 2 may have, in the so-called “closed” position, an external diameter which is identical to the external diameter of the main body of said guide pin.

Each blade 21, 22 also comprises a beveled end profile which facilitates the introduction of the guide pin 2 into the guide tube 3.

In the so-called “closed” position, the blades 21, 22 are separated from each other by a slot 24 ensuring that the bifid end 20 has a certain elasticity in order to allow the insertion of the guide pin 2 inside the guide tube 3.

FIGS. 3 to 7 show the various steps for placing the secured guiding device 1 according to the present invention in a bone element, for example a vertebra Ve for fixing a pedicle screw 4.

In order to place the guide pin 2, it is necessary beforehand to insert into the body of the vertebra Ve a straight cannula 5 passing through the cortical bone and positioning itself in the cancellous bone of the latter at a predetermined depth (FIG. 3).

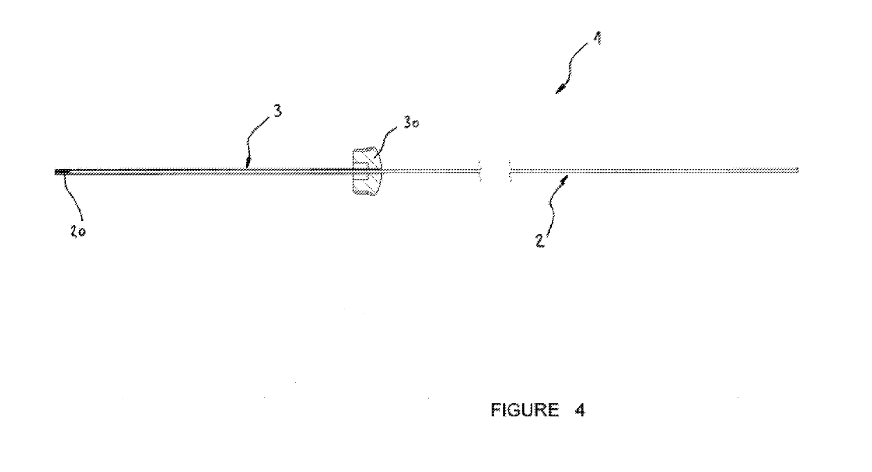

Then, the guide pin 2 is introduced into the guide tube 3 manually after bringing the blades 21, 22 of the bifid end 20 towards each other so as to keep them in the so-called “closed” position (FIG. 4).

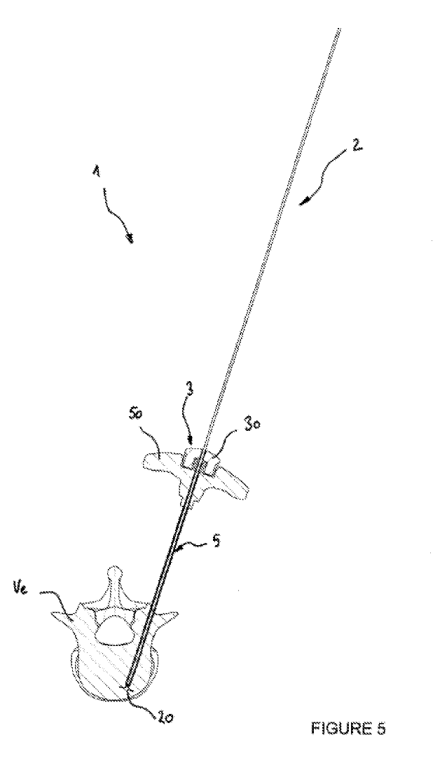

The guide tube 3 and the guide pin 2 are inserted inside the straight cannula 5 until the head 30 of said tube is pushed into abutment on the gripping head 50 of said straight cannula (FIG. 5).

The position of the straight cannula 5 in the vertebral body of the vertebra Ve determines the position of the guide pin 2 in the latter.

Indeed, since the guide tube 3 rests on the straight cannula 5, it suffices to push the guide pin 2 outside said guide tube so that the guide pin unfolds because of its elastic material, and the blades 21, 22 deviate from the longitudinal axis of the said pin and end up resting in the cancellous bone (FIG. 5).

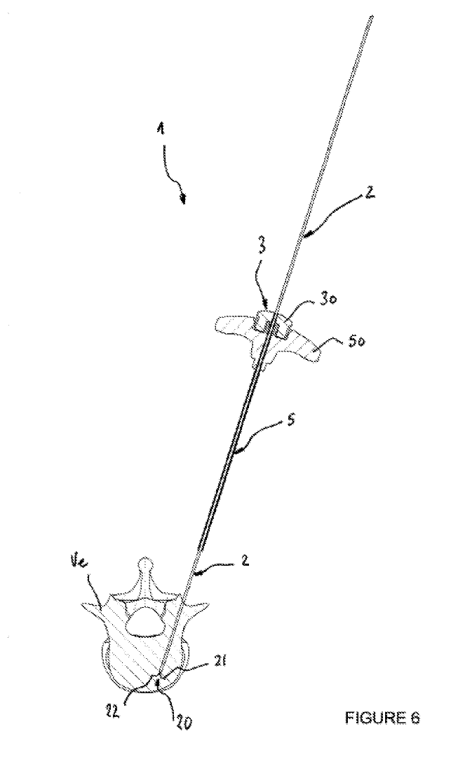

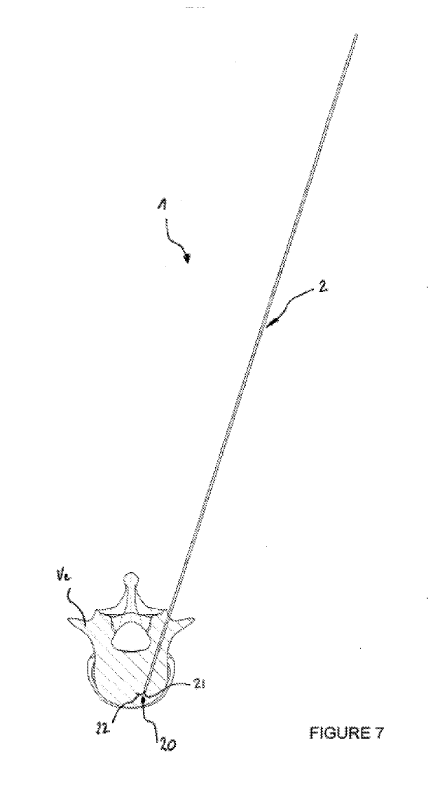

The straight cannula 5 and the guide tube 3 are removed from the vertebra Ve so as to only leave in place in said vertebra Ve the guide pin 2 of the secured guiding device 1 according to the present invention (FIGS. 6, 7).

FIGS. 8 and 9 illustrate the guiding of the pedicle screw 4 around the guide pin 2 of the secured guiding device 1 according to the present invention.

The pedicle screw 4 comprises an internal bore 40 opening out from one side to the other and enabling it to be placed around the guide pin 2. The pedicle screw 4 is screwed with the aid of a suitable screwdriver that cooperates with the clamping head 41 inside the body of the vertebra Ve.

When screwing the pedicle screw 4, the latter is guided axially inside the body of the vertebra Ve by means of the guide pin 2.

The pedicle screw 4 is considered to be in place in the vertebra Ve when its free end, opposite the clamping head 41, is located in the vicinity of the blades 21, 22 of the bifid end 20 of the guide pin 2.

Under a tensile force exerted on the guide pin 2, the blades 21, 22 of the bifid end 20 deform into a so-called “closed” position in order to penetrate under the stress in the internal bore 40 of the screw 4 and allow the removal of said guide pin 2.

The guide pin 2 has the advantage of opposing its own drive forward, that is to say, within the cancellous bone of the vertebra Ve, when tightening the pedicle screw 4, thus avoiding any risk of injury to the sensitive organs in the front of the vertebral body.

The guide pin 2 also limits the number of radiographic images and x-ray exposures to patients, surgeons and nursing staff present in the operating room.

It should moreover be understood that the foregoing description was given only by way of example and that it does not, in any way, limit the scope of the invention, and that replacing the details of execution described therein by any other equivalent would not fall outside the scope of the invention.

1. A secured guiding device for positioning and guiding a bone anchoring screw (4) during a surgical procedure, characterized in that it comprises a guide pin (2) having a bifid free end (20) which is elastically deformable and consists of two blades (21, 22) having, on the one hand, a first so-called “closed” configuration obtained, for example, by a manual stress so as to position said blades (21, 22) in the prolongation of the longitudinal axis of said guide pin (2) (2) thus allowing the latter to be inserted into a guide tube (3) for its positioning on the surgical site and, on the other hand, a second so-called “open” configuration in order to position said blades (21, 22) in two directions opposite and substantially perpendicular to that of the longitudinal axis of said guide pin (2) when the latter are outside the guide tube (3) in order to ensure a bone support when the anchoring screw (4) is being positioned.

2. The secured guiding device according to claim 1, characterized in that the blades (21, 22) of the guide pin (2) have, in the so-called “open” position, a radius of curvature with respect to the longitudinal axis of between 2 and 3 millimeters.

3. The secured guiding device according to claim 1, characterized in that the free ends of each blade (21, 22) of the guide pin (2) are in the so-called “open” position, spaced at a distance of between 8 and 12 millimeters.

4. The secured guiding device according to claim 1, characterized in that the free ends of each blade (21, 22) of the guide pin (2) respectively comprise on their outer radius a flat spot that provides a bone support.

5. The secured guiding device according to claim 1, characterized in that the guide pin (2) is made of a super-elastic alloy of the Nitinol type.

6. The secured guiding device according to claim 1, characterized in that the guide pin (2) has an outside diameter of less than 1.60 millimeters.

7. The secured guiding device according to claim 6, characterized in that the guide pin (2) has a bifid end (20) whose outer diameter, in the so-called “closed” position, is smaller than that of said guide pin.
